(19) United States
(12) Patent Application Publication
(10) Pub. No.: US 2010/0297225 A1
(43) Pub. Date: Nov. 25, 2010

(54) SUSTAINED-RELEASE PHARMACEUTICAL FORMULATION CONTAINING AN ANTIMUSCARINIC AGENT AND A WETTING AGENT AS WELL AS A PROCESS FOR THE PREPARATION THEREOF

(75) Inventors: Evangelos Karavas, Pallini Attikis (GR); Efthimios Koutris, Pallini Attikis (GR); Ioanna Koutri, Pallini Attikis (GR); Vicky Samara, Pallini Attikis (GR); Dimitrios Bikiaris, Thessaloniki (GR)

Correspondence Address: AKC PATENTS, 215 GROVE ST., NEWTON, MA, 02466, US

(73) Assignee: PHARMATHEN S.A., PALLINI-ATTIKIS (GR)

(21) Appl. No.: 12/747,211

(22) PCT Filed: Dec. 20, 2007
(86) PCT No.: PCT/EP2007/011230

§ 371 Date: Jul. 28, 2010

(51) Int. Cl. A61K 9/52; A61K 31/137; A61P 13/00

(52) U.S. Cl. 424/457; 514/648

(57) ABSTRACT

The present invention relates to improved sustained release dosage forms such as tablets and capsules, and in particular to a pharmaceutical formulation for oral administration comprising a therapeutically effective quantity of an antimuscarinic agent, such as Tolterodine or pharmaceutical acceptable salt, derivative, prodrug and metabolite thereof in combination with a wetting agent, such as Sodium Docusate to improve the release of the active ingredient and a method for the preparation thereof.

### TECHNICAL FIELD OF THE INVENTION

The present invention relates to improved sustained release dosage forms such as tablets and capsules, and in particular to a pharmaceutical formulation for oral administration comprising a therapeutically effective quantity of an antimuscarinic agent, and more particularly Tolterodine or pharmaceutical acceptable salt, derivative, prodrug and metabolite thereof in combination with a wetting agent such as Sodium Ducasate and a method for the preparation thereof.

### BACKGROUND OF THE INVENTION

Urinary incontinence is the involuntary excretion of urine from one's body. It is assumed that unstable or overactive bladder is caused by uncontrolled contractions of the bundles of smooth muscle fibers, forming the muscular coat of the urinary bladder, during its filling phase. The pharmacological treatment in such cases is the administration of muscarinic receptor antagonists such as Oxybutynin and Tolterodine.

Tolterodine is the (R)—N,N-diisopropyl-3-(2-hydroxy-5-methylphenyl)-3-phenylpropylamine and is an antimuscarinic drug that is used to treat urinary incontinence and other symptoms of unstable or overactive urinary bladder. It acts on M2 and M3 subtypes of muscarinic receptors whereas most antimuscarinic agents only act on M3 receptors. Tolterodine targets the bladder more than other areas of the body thus lower dose needs to be given daily (due to efficient targeting) and so causing fewer side effects.

Nevertheless, commercially sold immediate release formulations of Tolterodine are associated with side effects such as dry mouth, dyspepsia, headache due to high concentration of the drug in a short period of time. In order to overcome this problem extended release formulations have been developed. Extended release dosage forms are widely used for the administration of a variety of drugs since they maintain substantially constant blood levels and avoid the fluctuations associated with the immediate release formulations. Further, the extended release formulations provide many advantages over the immediate release dosage forms, such as increased patients compliance, improved delivery efficiency, decreased total drug requirement and minimization or even elimination of local or systemic side effects.

Furthermore, the dissolution profile of sustained release pharmaceutical compositions containing an antimuscarinic agent, and in particular Tolterodine or a pharmaceutical acceptable salt, derivative, prodrug and metabolite thereof can also be influenced by the selection of the excipients, as the drug release rate is dependent from the gastrointestinal pH-value and/or ionic strength. It is favorable for a sustained release formulation to possess drug release rates independent or less dependent from the ionic strength and/or pH of the environment through out the whole gastrointestinal tract in order to achieve better treatment to a patient.

Various methods are already known for the industrial preparation of extended release oral dosage forms comprising an antimuscarinic agent, and in particular Tolterodine or a pharmaceutical acceptable salt, derivative, prodrug and metabolite thereof as an active ingredient due to its useful therapeutical properties. However, the prior art has encountered substantial difficulties in the production of the oral solid formulations of a desirable dissolution profile and a cost effective manufacturing process.

EP 1 128 819 discloses a formulation containing controlled release beads comprising a core unit of a substantially water soluble or water swellable inert material, a first layer on the core of a substantially water insoluble polymer, a second layer over the first that contains an active ingredient and a third layer of polymer on the second layer effective for controlled release of the active ingredient, wherein the first layer is adapted to control water penetration into the core. This process is very complex, not cost effective and time consuming.

WO 2006/21425 discloses a composition comprising a core coated with an outer layer of a hydrophobic sustained release polymer. Said core is either a matrix core made of a matrix core material, Tolterodine and a binder or an inert core being provided with a layer of Tolterodine and a binder.

Although each of the above patents represents an attempt to overcome the problems associated with pharmaceuticals compositions for sustained release comprising an antimuscarinic agent, there still exists a need to provide a stable sustained release composition without producing unwanted pharmaceutical side effects and with improved release rate and low production costs.

### SUMMARY OF THE INVENTION

It is, therefore, an object of the present invention to provide an improved sustained release solid dosage formulation for oral administration containing an antimuscarinic agent, and in particular Tolterodine or pharmaceutical acceptable salt, derivative, prodrug and metabolite thereof as an active ingredient, which overcomes the deficiencies of the prior art.

It is another object of the present invention to provide a stable sustained release solid pharmaceutical dosage formulation for oral administration containing an antimuscarinic agent, and in particular Tolterodine or pharmaceutical acceptable salt, derivative, prodrug and metabolite thereof as an active ingredient, which is bioavailable and effective with sufficient self-life, good pharmacotechnical properties, enhancing patient compliance and reducing possible side effects.

Another aspect of the present invention is to provide a sustained release solid dosage formulation for oral administration containing an antimuscarinic agent, and in particular Tolterodine or pharmaceutical acceptable salt, derivative, prodrug and metabolite thereof as an active ingredient, having predictable and reproducible drug release rates independent or less dependent from the ionic strength and/or pH of the environment.

Moreover, another aspect of the present invention is to provide a sustained release solid dosage formulation for oral administration containing an antimuscarinic agent, and in particular Tolterodine or pharmaceutical acceptable salt, derivative, prodrug and metabolite thereof as an active ingredient, which can be prepared in dosage forms of different strength by proportionally adjusting the quantities of the excipients and the active ingredient, thereby providing a pharmacotechnical linearity, without affecting the dissolution profile and bioavailability of the active ingredient.

A further aspect of the present invention is to provide a method for the preparation of a sustained release solid dosage formulation for oral administration containing an antimuscarinic agent, and in particular Tolterodine or pharmaceutical acceptable salt, derivative, prodrug and metabolite thereof as an active ingredient, thereby enhancing the release rate of the active ingredient and being stable over a long period of time and improving the pharmacotechnical characteristics of the composition.

In accordance with the above objects of the present invention, a sustained release pharmaceutical composition for oral administration is provided comprising an antimuscarinic agent, and in particular Tolterodine or pharmaceutical acceptable salt, derivative, prodrug and metabolite thereof as an active ingredient, and an effective amount of a wetting agent, such as sodium docusate as an agent to improve the release of the active ingredient.

According to another embodiment of the present invention, a sustained release pharmaceutical composition for oral administration is provided comprising a hard gelatine capsule with a therapeutically effective number of mini tablets, said mini tablets comprising an antimuscarinic agent, and in particular Tolterodine or pharmaceutical acceptable salt, derivative, prodrug and metabolite thereof as an active ingredient, incorporated in a matrix of a water soluble and a water insoluble polymer and an effected amount of a wetting agent such as sodium docusate, as an agent to improve the release of the active ingredient.

According to another embodiment of the present invention, a process for the preparation of a sustained release solid dosage forms for oral administration such as tablets, capsules and sachets, containing an antimuscarinic agent, and in particular Tolterodine or pharmaceutical acceptable salt, derivative, prodrug and metabolite thereof as an active ingredient and an effective amount of a wetting agent such as Sodium Docusate as an agent to improve the release of the active ingredient is provided, which comprises:
dissolving the total quantity of said wetting agent such as sodium ducase into a solvent;
forming a homogenous mixture by mixing the total quantity of said active ingredient with the total quantity of a water insoluble polymer;
kneading the above mixture with the wetting agent solution;
adding to the formed solution the total quantity of a water soluble polymer and the total quantity of at least one optional excipient such as a diluent, a binder, a disintegrant, a glidant, a lubricant and wet granulating;
drying the wetted mass;
sieving the dried mass and adding to the sieved mixture the total quantities of at least one optional excipient such as a binder, a diluent, a disintegrant, a lubricant and/or a glidant and mixing until uniform, and
formulating the resulting mixture in a solid dosage form either by compressing it into a desired tablet form or by filling capsules or sachets.

Further preferred embodiments of the present invention are defined in dependent claims 2 to 10 and 12 to 14.

Other objects and advantages of the present invention will become apparent to those skilled in the art in view of the following detailed description.

### BRIEF DESCRIPTION OF THE DRAWINGS

FIG. 1 shows X-RD spectrums of the pharmaceutical composition 1 according to the present invention directly after preparation and after three months of storage in normal and accelerated conditions.

FIG. 2 shows dissolution profiles of the pharmaceutical composition 1 according to the present invention with different quantities of sodium docusate in buffer pH 6.8.

FIG. 3 shows average plasma value for the composition 1 of Example 1 according to the present invention and a known marketed product.

FIG. 4 shows dissolution profiles of the pharmaceutical composition 1 according to the present invention in buffer pH 6.8 and changes of pH from 1.2 to 6.8.

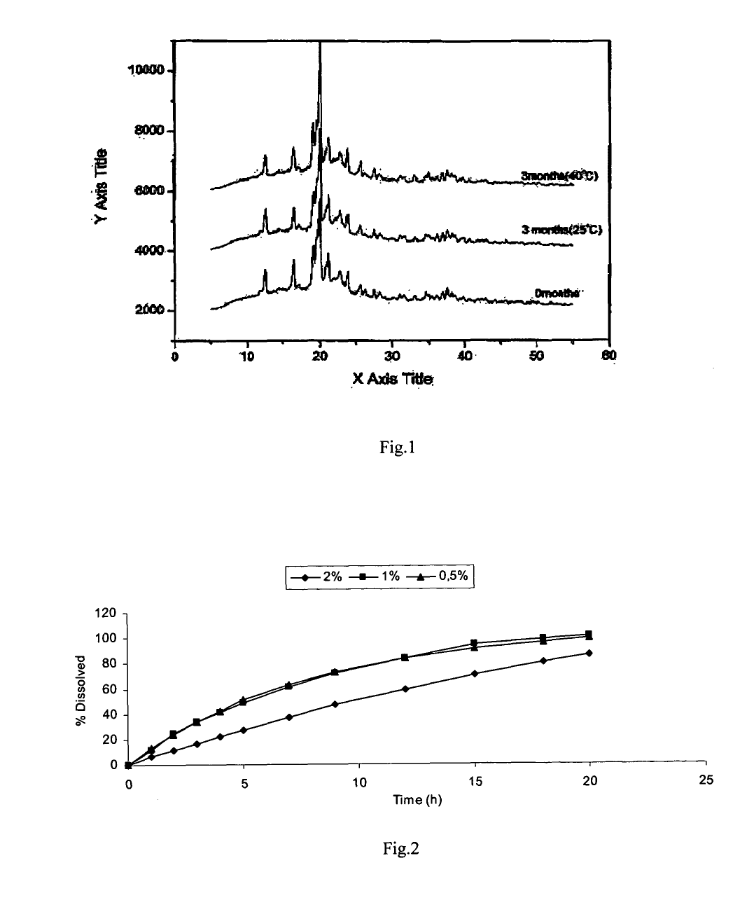

### DETAILED DESCRIPTION OF THE INVENTION

For the purposes of the present invention, a pharmaceutical composition comprising an antimuscarinic agent (i.e. Tolterodine or pharmaceutical acceptable salt, derivative, prodrug and metabolite thereof) is considered to be “stable” if said ingredient degradates less or more slowly than it does on its own and/or in known pharmaceutical compositions.

The active ingredient (an antimuscarinic agent i.e. Tolterodine or pharmaceutical acceptable salt, derivative, prodrug and metabolite thereof) contained in a dosage form is “bioavailable”, if when administered in a dosage form is released from the dosage form, absorbed and reaches, at least the same, concentration levels in plasma as any of the marketed products containing the same quantity of the same active ingredient and intended for the same use.

An excipient is considered to be “incompatible” with an active ingredient (an antimuscarinic agent i.e. Tolterodine or pharmaceutical acceptable salt, derivative, prodrug and metabolite thereof) if it promotes the degradation of said active ingredient, that is to say, if said active ingredient degrades more or faster in the presence of said excipient when compared with the degradation of said active ingredient on its own. The terms “incompatibility”, “compatible” and “compatibility” are defined accordingly.

The term “pharmaceutically acceptable” refers to a form that is acceptable to the patient from a pharmacological or toxicological point of view, including acceptable composition, formulation, stability, bioavailability and acceptance by the patient.

Although the pharmaceutical composition may be in various forms, the preferred solid forms are tablets, capsules and caplets.

As already mentioned the main object of the present invention is to provide a sustained release formulation of Tolterodine or pharmaceutical acceptable salt, derivative, prodrug and metabolite thereof that is simple to manufacture, cost effective, posses good pharmacotechnical properties and linearity.

It has been surprisingly found that the object of the present invention is achieved by employing an effective amount of a wetting agent, such as Sodium Docusate in order to improve the release of the active ingredient.

Sodium Docusate is widely used as anionic surfactant and wetting agent in pharmaceutical formulations. Docusate salts are also used in oral formulations as laxatives and fecal softeners. When Sodium Docusate is incorporated in a pharmaceutical composition according to the present invention, it acts as a carrier and forms inverse micelles that entrap the active ingredient and delay the release of the drug substance.

The active ingredient (antimuscarinic agent such as Tolterodine or pharmaceutical acceptable salt, derivative, prodrug and metabolite thereof) and a suitable amount of water insoluble polymer are being admixed and subsequently wet granulated with a Sodium Docusate solution.

A suitable amount of water soluble polymer and an optional excipient is being added in the solution. After drying the wetted mass and sieving, any optional additional excipient is then added. The composition is then mixed until uniform. The resulting composition may then be compressed.

The matrix formation according to the present invention comprises both water soluble and water insoluble polymers. When a dosage form according to the present invention is introduced into gastric or intestinal fluid, the water soluble polymers leach out from the matrix to form pores through which the active ingredient slowly diffuses outwards.

However, the composition according to the present invention exhibits more predictable and reproducible drug release rate than a regular matrix. This is attributed to the characteristics of Sodium Docusate and its interaction with the active ingredient.

Moreover, any excipient may optionally be added to the above composition, provided that they are compatible with the active ingredient of the composition, in order to overcome problems associated with the unfavorable pharmacotechnical characteristics of these substances, and in order to increase the stability of the drug and the self-life of the pharmaceutical product, and provide a product exhibiting excellent bioavailability.

The present invention can be applied in the formulation of tablets, capsules, caplets, sachets or other solid dosage forms for oral administration of an active ingredient.

Furthermore, it is possible to prepare dosage forms of different strength using appropriate quantity of the same composition, thereby limiting the cost of production and minimizing the number, and consequently the cost, of clinical studies required for the approval of the product by the authorities.

The manufacturing process for the preparation of the composition according to the present invention is simpler and inexpensive in comparison to any other conventional method.

In the present invention, a sustained release medicament in the form of a hard gelatin capsule filled with mini-tablets is provided. The use of mini-tablets is beneficial because pharmacotechnical linearity between the strength and the formulation is achieved easily by incorporating one or more of the mini-tablets in the capsule. Linearity is highly desired in the pharmaceutical industry for manufacturing, pharmacokinetic and economical reasons.

Tabletting was the chosen production method because it is faster, easier, adds fewer steps to the process and is the most economical. Further, the tableting method ensures a high production yield, contrary to the manufacture of pellets where the loss of production output is usually much higher. Excipients for the formulation were chosen carefully to give appropriate dissolution rate and stability of the finished dosage form.

In a first embodiment, the present invention provides a pharmaceutical composition comprising from about 0.5% to 50% by weight of Tolterodine or salt thereof and from about 0.05% to 2% by weight of Sodium Docusate. The weight ratio of the Tolterodine or pharmaceutical acceptable salt, derivative, prodrug and metabolite thereof to Sodium Docusate is preferably 1:4 to 1000:1.

Preferred pharmaceutical compositions according to the present invention comprise Tolterodine or salt thereof in an amount approximately 0.5% to 40%, more preferably 0.5% to 25% and most preferably 0.5% to 15%.

More preferred pharmaceutical compositions according to the present invention comprise Sodium Docusate in an amount approximately 0.1% to 2%, and most preferably 0.1% to 1.5%.

The preferred pharmaceutical compositions are in the form of solid dosage forms for oral or sub-lingual administration such as tablets, capsules, caplets, troches, pastilles, pills, lozenges and the like, in all shapes and sizes, coated or uncoated.

All percentages stated herein are weight percentages based on total composition weight, unless otherwise stated.

The preferred pharmaceutical compositions are in the form of solid dosage forms for oral or sub-lingual administration such as tablets, capsules, caplets, troches, pastilles, pills, lozenges and the like, in all shapes and sizes, coated or uncoated.

All percentages stated herein are weight percentages based on total composition weight, unless otherwise stated.

Another embodiment of the present invention is the use of the wet granulation process for the preparation of a sustained release solid dosage forms for oral administration such as capsules containing minitablets comprising an antimuscarinic agent, and in particular Tolterodine or pharmaceutical acceptable salt, derivative, prodrug and metabolite thereof as an active ingredient and an effective amount of a wetting agent such as Sodium Docusate as an agent to improve the release of the active ingredient. Said wet granulation process comprises:
dissolving the total quantity of said wetting agent such as sodium ducase in water;
forming a homogenous mixture by mixing the total quantity of said active ingredient with the total quantity of a water insoluble polymer;
kneading the above mixture with the wetting agent solution;
adding to the formed solution the total quantity of a water soluble polymer and the total quantity of at least one optional excipient such as a diluent, a binder, a disintegrant, a glidant, a lubricant and wet granulating;
drying the wetted mass;
sieving the dried mass and adding to the sieved mixture the total quantities of at least one optional excipient such as a binder, a diluent, a disintegrant, a lubricant and/or a glidant and mixing until uniform, and
formulating the resulting mixture in a solid dosage form either by compressing it into a desired minitablet form and/or by filling capsules.

The pharmaceutical compositions of the present invention may also contain one or more additional formulation ingredients selected from a wide variety of excipients. According to the desired properties of the composition, any number of ingredients may be selected, alone or in combination, based upon their known uses in preparation of solid dosage form compositions.

Such ingredients include, but are not limited to, diluents, binders, compression aids, disintegrants, surfactants, antioxidants, glidants, lubricants, flavors, water scavengers, colorants, sweetener, coating agents and preservatives.

Diluents may be, for example, calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol sorbitol, starch, pregelatinized starch, sucrose, talc, xylitol, maltose maltodextrin, maltitol.

Binders may be, for example, acacia mucilage, alginic acid, carbomer, carboxymethylcellulose calcium, carboxymethylcellulose sodium, microcrystalline cellulose, powdered cellulose, ethyl cellulose, gelatin, liquid glucose, guar gum, hydroxyethyl cellulose, hydroxypropyl cellulose, hydroxypropylmethyl cellulose, maltodextrin, methylcellulose, polydextrose, polyethylene oxide, povidone, sodium alginate, starch paste, pregelatinized starch, sucrose.

Disintegrants may be, for example, alginic acid, carbon dioxide, carboxymethylcellulose calcium, carboxymethylcellulose sodium, microcrystalline cellulose, powdered cellulose, croscarmelose sodium, crospovidone, sodium docusate, guar gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch.

Surfactants may be, polyoxyethylene-polyoxypropylene co-polymers and block co-polymers, commercially available as Pluronic@ or Poloxamer@, ethoxylated cholesterins, commercially available as Solulan@, vitamin derivatives, e.g. vitamin E derivatives such as tocopherol polyethylene glycol succinate (TPGS), sodium dodecylsulfate or sodium laurylsulfate; —a bile acid or salt thereof, for example cholic acid, glycolic acid or a salt.

Glidants may be, for example, calcium silicate, powdered cellulose, starch, talc, colloidal silicon dioxide.

Lubricants e.g. polyethylene glycol 4000, polyethylene glycol 6000, sodium lauryl sulfate, starch, talc.

One or more suitable water soluble polymers may be selected to be used in the invention, which include, but are not limited to, cellulose derivatives (e.g. hydroxypropylmethylcellulose, hydroxypropylcellulose, methylcellulose, etc.), alginic acid, propylene glycol, pectin, low methoxy pectin, guar gum, gum arabic, carrageenan, xanthan gum, synthetic water-soluble polymer (e.g. polyvinylpyrrolidone, etc.) and the like.

One or more suitable water insoluble polymers may be selected to be used in the invention, which include, but are not limited to, cellulose derivatives (e.g. ethylcellulose), polyvinyl acetate (Kollidon SR from BASF), neutral copolymers based on ethyl acrylate and methylmethacrylate, copolymers of acrylic and methacrylic acid esters with quaternary ammonium groups, such as Eudragit NE, RS or RS30D, RL or RL30D and the like.

The following examples illustrate preferred embodiments in accordance with the present invention without limiting the scope or spirit of the invention:

### EXAMPLES

### Example 1

### Tolterodine Composition 1

| Ingredients | % by weight |
|---|---|
| Tolterodine Tartrate | 2.50 |
| Microcellac | 56.00 |
| Kollidon SR | 10.00 |
| Methocel K100M | 30.00 |
| Sodium Docusate | 1.00 |
| Mg Stearate | 0.50 |
| Total | 100.00 |

Minitablets of the above formulation were prepared according to the following manufacturing process: Sodium Docusate was dissolved in water. Tolterodine Tartrate was mixed with Kollodin SR to form a homogenous mixture. The above mixture was kneaded with the solution of Sodium Docusate. Microcellac and Methocell K100M were added and wet granulated. The granular mass was dried. Finally Mg Stearate was added to the dried granule and mixed until complete homogeneity. The resulting granule was compressed into minitablets and filled into capsules.

The pharmaceutical composition is characterized by excellent pharmacotechnical properties, such as homogeneity, flowability and compressibility. Namely, the pure pharmaceutical substance Tolterodine tartrate showed limited flowability and compressibility with a mean Carr's Index of 38.7%. However, when a lubricant was incorporated, the Carr Index observed was 23.2%, which indicates an improvement of the flow properties of Tolterodine final formulation.

Another object of the present invention was to prepare a pharmaceutical composition that is stable for a long period of storage time. Therefore, capsules of composition 1 were exposed to normal (25° C.±2° C./60% ±5% RH) and accelerated (40° C.±2° C./75% ±5% RH) stability studies according to the current ICH guidelines.

The stability results are shown in TABLE 1 below.

TABLE 1
Stability results of composition 1 directly after preparation and
after 3 months of storage in normal and accelerated conditions

%

3 Months

| IMPURITIES | 0 Months | Normal | Accelerated |
|---|---|---|---|
| Imp 1 | ND | ND | ND |
| Imp 4 | 0.04% | 0.06% | 0.07% |
| Unknown | 0.14% | 0.16% | 0.16% |
| Unknown | 0.05% | 0.07% | 0.08% |
| Total | 0.23% | 0.29% | 0.31% |

As it is indicated, the stability of the present invention is good since the impurity content after three months remains in a low range.

According to another aspect of the present invention, the active substance should remain in the same state after compression and should not convert in another form.

As shown in FIG. 1 by the X-RD analysis Tolterodine Tartrate is in crystalline form and all the recorded characteristic broad peaks (approximate 20 values=12.55, 16.40, 20.00, 21.20, 22.75, 23.80, 25.65, 27.45, 34.60 and 37.60) were unchanged after 3 months storage in both normal and accelerated conditions (40° C. and 75% RH). The crystal properties remain also unchanged after three months in the same conditions when the mixture is incorporated in a pharmaceutical composition with other excipients. No peaks corresponding to another crystalline form of Tolterodine Tartrate are observed before or after storage indicating that the mixture is stabilized.

The bioavailability and pharmacokinetic profile of composition 1 of the present invention was determined in “in vivo” single-dose study.

A single-dose study was conducted in 12 healthy volunteers using a formulation prepared with crystalline form Tolterodine Tartrate according to Example 1. The reference compound was a 4 mg Tolterodine Tartrate capsule that consists of the active ingredient, sucrose, starch, hypromellose, ethylcellulose, medium chain triglycerides, oleic acid, gelatin, and FD&C Blue #2 (Composition B). Plasma samples were removed and tested for Tolterodine.

Table 2 shows the main pharmacokinetic parameters obtained from the test.

TABLE 2
Pharmacokinetic analysis of composition
1 versus reference composition B

| | lnAUC0-t | lnAUC0-inf | Cmax |
|---|---|---|---|
| | (ng h/ml) | (ng h/ml) | (ng/ml) |
| Ratio of least square means % | 110.7 | 110.4 | 110.5 |
| 90% C.I. % | 98-124.9 | 97.1-125.4 | 91.7-133 |
| Intra subject CV % | 16.5 | 17.4 | 25.4 |

wherein:
C max = (peak concentration) is the highest concentration reached by the drug in plasma after dosing;
AUC₀₋ₜ = (area under the curve) The area under the plasma concentration versus time curve, from time 0 to the last measurable concentration, as calculated by the linear trapezoidal method.
AUC_{0-inf} = (area under the curve) The area under the plasma concentration versus time curve from time 0 to infinity. AUCinf is calculated as the sum of AUC 0-t plus the ratio of the last measurable plasma concentration to the elimination rate constant.

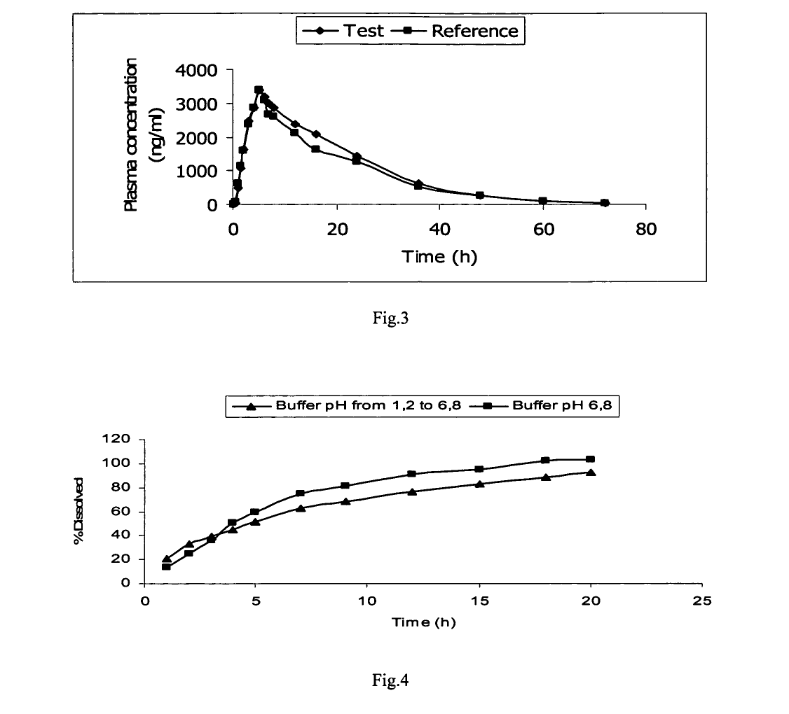

Consequently, it has been found that composition 1 (4 mg capsule) has an adequate bioavailability compared to the reference composition B (FIG. 3).

Another object of the present invention was to provide a sustained release composition with predictable and reproducible drug release rates independent or less dependent of the ionic strength and/or pH of the environment.

Composition 1A, 1B and 1C, as illustrated in TABLE 3 below, with different quantitites of Sodium Docusate were tested (2%, 1% and 0.5%). The dosage forms of said compositions 1A, 1B and 1C were prepared according to the manufacturing process of composition 1

TABLE 3
Composition of examples 1A, 1B and 1C

| | % Content | | |
|---|---|---|---|
| Ingredients | Comp. 1A | Comp. 1B | Comp. 1C |
| Tolterodine Tartrate | 2.50 | 2.50 | 2.50 |
| Microcellac | 35.00 | 36.00 | 36.50 |
| Kollidon SR | 10.00 | 10.00 | 10.00 |
| Methocel K100M | 50.00 | 50.00 | 50.00 |
| Sodium Docusate | 2.00 | 1.00 | 0.50 |
| Mg Stearate | 0.50 | 0.50 | 0.50 |
| Total | 100.00 | 100.00 | 100.00 |

According to the dissolution results, the decrease of Sodium Docusate proportion from 2% to 1% showed a significant change in the drug release rate, but the respective decrease from 1% to 0.5% showed no significant change (FIG. 4). Therefore, the present invention provides a pharmaceutical formulation comprising Sodium Docusate in a quantity of less than 2% of the total weight of the composition.

The dissolution profiles of compositions 1A, 1B and 1C are presented in TABLE 4 below.

TABLE 4
Dissolution profiles of compositions 1A, 1B,
and 1C in phosphate buffered saline pH 1.2,
change pH from 1.2 to 6.8 and buffer pH 6.8.

| | % Dissolved | | % Dissolvde | | | % Dissolv | | |
|---|---|---|---|---|---|---|---|---|
| | (buffer pH 1.2) | | (buffer Ph 1.2 to 6.8) | | | (buffer pH 6.8) | | |
| time | Ex. | Ex. | Ex. | Ex. | Ex. | Ex. | Ex. | Ex. |
| (h) | 1A | 1B | 1A | 1B | 1C | 1A | 1B | 1C |
| 1 | 8.11 | 14.17 | 8.99 | 15.47 | 18.03 | 6.37 | 11.83 | 13.02 |
| 2 | 14.47 | 24.68 | 16.15 | 26.30 | 31.54 | 11.38 | 25.16 | 24.12 |
| 3 | 20.51 | 34.31 | 20.44 | 30.79 | 37.69 | 16.83 | 33.95 | 34.13 |
| 4 | 26.94 | 43.25 | 23.98 | 36.65 | 44.44 | 22.22 | 41.76 | 41.91 |
| 5 | 32.51 | 51.31 | 27.19 | 41.93 | 50.21 | 27.44 | 49.11 | 51.05 |
| 7 | 43.81 | 65.09 | 33.79 | 51.92 | 60.59 | 37.39 | 61.20 | 62.52 |
| 9 | 53.75 | 75.93 | 40.20 | 59.02 | 68.66 | 46.77 | 72.24 | 72.50 |
| 12 | 67.07 | 88.07 | 48.88 | 69.12 | 77.05 | 58.50 | 83.75 | 83.52 |
| 15 | 77.73 | 96.57 | 56.29 | 77.18 | 82.52 | 70.05 | 94.18 | 91.24 |
| 18 | 86.46 | 102.92 | 62.78 | 81.30 | 87.58 | 80.33 | 98.15 | 95.75 |
| 20 | 93.10 | 106.41 | 64.72 | 86.21 | 90.53 | 86.16 | 101.26 | 99.27 |

The dissolution profiles of composition 1 (comprising 1% of Sodium Docusate) are presented in TABLE 5.

TABLE 5
Dissolution profiles of composition 1 in phosphate buffered
saline pH 6.8 and changes of pH from 1.2 to 6.8.

| | % Dissolved | % Dissolved |
|---|---|---|
| time | (buffer pH 1.2 to 6.8) | (buffer pH 6.8) |
| (h) | Comp. 1 | Comp. 1 |
| 1 | 20.84 | 13.94 |
| 2 | 33.06 | 24.70 |
| 3 | 39.47 | 36.58 |
| 4 | 45.25 | 50.51 |
| 5 | 51.57 | 59.99 |
| 7 | 62.59 | 75.25 |
| 9 | 68.74 | 81.19 |
| 12 | 76.49 | 91.09 |
| 15 | 83.22 | 95.30 |
| 18 | 88.60 | 102.29 |
| 20 | 92.95 | 103.16 |

The dissolution results in connection with the data as depicted from FIG. 3 indicate the effectiveness of the present invention in providing predictable and reproducible drug release rates, independent or less dependent of the ionic strength and/or pH of the environment.

### Example 2

### Tolterodine Comnositinn 2

| Ingredients | % Content |
|---|---|
| Tolterodine Tartrate | 2.50 |
| Microcellac | 47.00 |
| Gelcarin GP-379NF | 40.00 |
| PVP | 10.00 |
| Mg Stearate | 0.50 |
| Total | 100.00 |

Mini-tablets incorporated in a hard gelatin capsule have been prepared according to the composition 2.

The combination of two polymers, povidone (PVP) which is used widely as a suspending and viscosity-increasing agent and an iota Carrageenan, Gelcarin GP-379NF, which is a gelling polymer, were tested for the matrix formation. The manufacturing process used was direct compression, beginning with geometrical mixing of Tolterodine Tartrate with the polymers (PVP and Gelcarin), in order to achieve uniformity of content. The total weight of each minitablet in this formulation step was determined to be 80 mg. Subsequently, 4 mg potency of the capsules was prepared, each capsule containing two tablets of 80 mg weight (equivalent to 2 mg/tablet of Tolterodine each), resulting to 160 mg in total weight.

The dissolution profile given from the dissolution test of the capsules in dissolution medium phosphate buffered saline pH 6.8, 100 rpm is shown in TABLE 6 below.

TABLE 6
Dissolution results of composition 2
in phosphate buffered saline pH 6.8

| Time | % Dissolved |
|---|---|
| (h) | Ex. 2 |
| 1 | 95.00 |
| 2 | 95.34 |
| 3 | 96.30 |
| 4 | 96.55 |
| 5 | 96.66 |
| 7 | 96.87 |
| 9 | 96.71 |
| 12 | 96.54 |
| 15 | 94.85 |

From the above results it is obvious that the drug release of composition 2 should be suppressed significantly and thus does not satisfy the requirements of the present invention.

### Example 3

### Tolterodine Composition 3

| | % Content |
|---|---|
| Ingredients | Ex. 3 |
| Tolterodine Tartrate | 2.50 |
| Microcellac | 27.00 |
| Viscarin GP 209NF | 55.00 |
| Gelcarin GP-379NF | 15.00 |
| Mg Stearate | 0.50 |
| Total | 100.00 |

The combination of two Carrageenans, iota Carrageenan, Gelcarin GP-379NF, with the lambda Carrageenan, Viscarin GP-209NF, was tested for the matrix formation. A number of Tolterodine Tartrate capsules filled with direct compression mini-tablets were produced according to the composition 3.

The dissolution profile of composition 3 of example 3 is presented in TABLE 7 below.

TABLE 7
Dissolution results of composition 3 of example 3 with changes
of pH from 1.2 to 6.8 and in phosphate buffered saline pH 6.8

| | | |
|---|---|---|
| | % Dissolved | % Dissolved |
| Time | (buffer pH 1.2 to 6.8) | (buffer pH 6.8) |

| | |
|---|---|
| (h) | Ex. 3 |

| | | |
|---|---|---|
| 1 | 18.67 | 14.28 |
| 2 | 33.79 | 29.44 |
| 3 | 45.17 | 42.56 |
| 4 | 56.79 | 54.52 |
| 5 | 65.41 | 65.18 |
| 7 | 82.35 | 81.15 |
| 9 | 94.60 | 91.18 |
| 12 | 100.81 | 95.96 |
| 15 | 101.85 | 101.70 |

The dissolution results of the above compositions indicate that the dissolution rate was decreased in comparison to composition 2. Nevertheless, the results are high in dissolution media buffer 1.2 to 6.8, probably due to fact that Gelcarin is a pH dependent polymer.

### Example 4

### Tolterodine Composition 4

| | % Content |
|---|---|
| Ingredients | Ex. 4 |
| Tolterodine Tartrate | 2.50 |
| Microcellac | 47.00 |
| Kollidon SR | 10.00 |
| Methocel K100M | 40.00 |
| Mg Stearate | 0.50 |
| Total | 100.00 |

The combination of Kollidon SR (a blend of polyvinyl acetate and povidone used as a matrix-forming agent) and Methocel K100M according to composition 4 was tested and tablets with direct compression process filled in hard gelatin capsules were prepared.

The dissolution profile of composition 4 in different dissolution mediums is presented in TABLE 8 below.

According to the dissolution results, Kollidon SR and Methocel K100M seemed to be more effective than the combination of carrageenans (composition 3), however the dissolution results of composition 4 in acidic conditions (buffer pH 1.2) were not satisfactory because the drug substance release was quick, as depicted from TABLE 8.

TABLE 8
Dissolution results of composition 4 of example 4 in phosphate
buffered saline pH 6.8 and in buffer pH 1.2.

| | | |
|---|---|---|
| | % Dissolved | % Dissolved |
| time | (buffer pH 1.2) | (buffer pH 6.8) |

| | |
|---|---|
| (h) | Ex. 4 |

| | | |
|---|---|---|
| 1 | 19.61 | 14.15 |
| 2 | 33.15 | 25.17 |
| 3 | 45.45 | 34.60 |
| 4 | 57.08 | 43.52 |
| 5 | 67.62 | 51.33 |
| 7 | 84.96 | 63.93 |
| 9 | 95.71 | 72.46 |
| 12 | 102.79 | 79.90 |
| 15 | 104.89 | 84.00 |
| 18 | 105.89 | 87.26 |
| 20 | 106.23 | 89.20 |

### Example 5

### Tolterodine Composition 5

| | % Content |
|---|---|
| Ingredients | Ex. 5 |
| Tolterodine Tartrate | 2.50 |
| Calcium Carbonate | 17.00 |
| Kollidon SR | 40.00 |
| Methocel K100M | 40.00 |
| Mg Stearate | 0.50 |
| Total | 100.00 |

In order to suppress the release in acidic conditions, the use of an alkaline agent, specifically calcium carbonate, as a diluent instead of Microcellac, was incorporated. Capsules filled with minitablets according to composition 5 were prepared using direct compression manufacruring process.

The dissolution profile of composition 5 is presented in TABLE 9 below.

Composition 5 possesses satisfactory in vitro results, however the in vivo results showed that the bioavailability achieved was not adequate.

TABLE 9
Dissolution results of composition 5 of example 5 in phosphate buffered
saline pH 6.8, change pH from 1.2 to 6.8 and buffer pH 1.2.

| | % Dissolved | % Dissolved | % Dissolved |
|---|---|---|---|
| Time | (buffer pH 1.2) | (buffer pH 1.2 to 6.8) | (buffer pH 6.8) |
| (h) | Ex. 5 | | |
| 1 | 11.63 | 12.24 | 11.79 |
| 2 | 18.57 | 19.86 | 19.74 |
| 3 | 25.34 | 26.13 | 26.80 |
| 4 | 30.75 | 31.19 | 32.88 |
| 5 | 35.57 | 37.17 | 39.13 |
| 7 | 45.29 | 47.63 | 49.27 |
| 9 | 54.47 | 56.85 | 57.55 |
| 12 | 64.73 | 68.65 | 67.78 |
| 15 | 72.03 | 77.62 | 75.82 |
| 18 | 76.65 | 83.12 | 82.18 |
| 20 | 79.46 | 86.80 | 85.90 |

The pharmaceutical formulation according composition 1 of the present invention has excellent pharmacotechnical properties indicating the suitability of the process and of the selected excipients as well.

Another essential advantage of the present invention is that it ensures excellent bioavailability of the active ingredient. Furthermore, it is possible to prepare dosage forms of different strength by adjusting the number of minitablets incorporated in the capsule, thereby limiting the cost of production and minimizing the number, and consequently the cost, of clinical studies required for the approval of the product by the authorities. The manufacturing process for preparation according to the present invention is simple and inexpensive in comparison to other conventional methods.

The stability of the product as well as the simple and economic manufacturing process indicates the advantages of the present invention relative to the commonly used methods and excipients for the formulation of Tolterodine. Also, the present invention provides a drug release that is independent or less dependent of the pH and/or the ionic strength of the dissolution medium.

Although the pharmaceutical composition may be in various forms, the preferred solid forms are tablets, capsules and caplets and more preferred incorporation of mini-tablets in a hard gelatin capsule.

While the present invention has been described with respect to the particular embodiments, it will be apparent to those skilled in the art that various changes and modifications may be made in the invention without departing from the spirit and scope thereof, as defined in the appended claims.

1. A sustained release pharmaceutical composition for oral administration comprising an antimuscarinic agent, such as Tolterodine or pharmaceutical acceptable salt, derivative, prodrug and metabolite thereof, as an active ingredient and an effective amount of a wetting agent such as Sodium Docusate to improve the release of the active ingredient.

2. The pharmaceutical composition according to claim 1, wherein said wetting agent is sodium docusate.

3. The pharmaceutical composition according to claim 2, wherein it comprises from about 0.5% to 50% by weight of said antimuscarinic agent or pharmaceutical acceptable salt, derivative, prodrug and metabolite thereof, and from about 0.05% to 2.0% by weight of said wetting agent.

4. The pharmaceutical composition according to claim 2, wherein the weight ratio of said antimuscarinic agent to the wetting agent is preferably 1:4 to 1000:1.

5. The pharmaceutical composition according to claim 1, wherein said antimuscarinic agent is Tolterodine or pharmaceutical acceptable salt, derivative, prodrug and metabolite thereof.

6. The pharmaceutical composition according to claim 1, further comprising at least one water insoluble polymer, such as Polyvinyl acetate and Povidone.

7. The pharmaceutical composition according to claim 1, further comprising at least one water soluble polymer, such as hydroxypropyl methyl cellulose.

8. The pharmaceutical composition according to claim 1, further comprising at least one optional excipient selected from the group consisting of diluents, binders, disintegrants, lubricants and glidants.

9. The pharmaceutical composition according to claim 1, further comprising lactose, microcrystalline cellulose, magnesium stearate, polyvinyl acetate, polyvinylpyrrolidone and hydroxypropyl methyl cellulose.

10. The pharmaceutical composition according to claim 1, wherein said composition is in solid dosage form such as tablet, capsule or sachet and more preferably mini tablets incorporated in a hard gelatin capsule.

11. A process for the preparation of a sustained release solid dosage form for oral administration such as a tablet, capsule or sachet comprising an antimuscarinic agent, such Tolterodine or pharmaceutical acceptable salt, derivative, prodrug and metabolite thereof as an active ingredient and an effective amount of a wetting agent such as Sodium Docusate to improve the release of the active ingredient, which process comprises:
dissolving the total quantity of said wetting agent such as sodium docusate in water;
forming a homogenous mixture by mixing the total quantity of said active ingredient with the total quantity of a water insoluble polymer;
kneading the above mixture with the wetting agent solution;
adding to the formed solution the total quantity of a water soluble polymer and the total quantity of at least one optional excipient such as a diluent, a binder, a disintegrant, a glidant, a lubricant and wet granulating;
drying the wetted mass;
sieving the dried mass and adding to the sieved mixture the total quantities of at least one optional excipient such as a binder, a diluent, a disintegrant, a lubricant and/or a glidant and mixing until uniform, and—formulating the resulting mixture in a solid dosage form either by compressing it into a desired tablet form and/or by filling capsules or sachets.

12. The process according to claim 11, wherein said wetting agent is sodium docusate.

13. The process according to claim 11, wherein said antimuscarinic agent is Tolterodine or pharmaceutical acceptable salt, derivative, prodrug and metabolite thereof.

14. The process according to claim 11, wherein the final mixture is being compressed into mini tablets and subsequently filled into hard gelatin capsule.
